(19) United States
(12) Patent Application Publication
(10) Pub. No.: US 2021/0338959 A1
(43) Pub. Date: Nov. 4, 2021

(54) CPAP MASK WITH SIDE PORTS FOR PROVIDING CONTINUOUS POSITIVELY PRESSURIZED AIR TO A PATIENT WITH RESPIRATORY DISTRESS AND OTHER RESPIRATORY MEDICAL CONDITIONS

(71) Applicant: Sonal Asthana, Gurgaon (IN)

(72) Inventors: Sonal Asthana, Haryana (IN); Ankit Agarwal, Haryana (IN); Shantha Kumar S.P, Haryana (IN)

(21) Appl. No.: 17/133,668

(22) Filed: Dec. 24, 2020

(30) Foreign Application Priority Data
May 1, 2020 (IN) 202011018640

(51) Int. Cl. A61M 16/06; A61M 16/10

(52) U.S. Cl. CPC A61M 16/0622; A61M 2205/3337; A61M 16/105

(57) ABSTRACT

The present disclosure provides a CPAP mask with side ports for providing continuous positively pressurized air to a patient. The CPAP mask includes a mask body, a plurality of strap holders, and a cushion seal. The mask body includes a left side wall, a left port, a front face, a right side wall, and a right port. In addition, the left side wall has a first through-hole. Further, the left port is on the left side wall. Furthermore, the front face of the CPAP mask is a transparent member. Moreover, the right side wall has a second through-hole. Also, the right port is on the right side wall. Also, the plurality of strap holders is on the left side wall and the right side wall.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

### Field of the Invention

The present disclosure relates to the field of respiratory devices and, in particular, relates to a Continuous Positive Airway Pressure (CPAP) mask with side ports for providing continuous positively pressurized air to a patient with respiratory distress or respiratory medical conditions.

### Description of the Related Art

Respiratory disorders are potentially life-threatening disorders characterized by brief interruptions of breathing. The respiratory disorders include asthma, chronic obstructive pulmonary disease (COPD), pulmonary fibrosis, sleep apnea, pneumonia, lung cancer, and the like. In addition, early recognition and treatment of the respiratory disorders is important. Further, positive airway pressure is an effective form of therapy for a patient suffering from a respiratory disorder where there is inflammation of the lung internal walls caused by COVID19 virus, SARS virus, MERS, Influenza virus and the like. Recently, it has been established by the International Medical Community that a Non-invasive form of respiratory support is most ideal and best treatment procedure to treat patients on a very large scale that the world is faced with due to COVID19 Pandemic. In addition, CPAP is the Non-invasive treatment and is now widely adopted in treating Patients affected with COVID19 Virus. Further, ventilation is applied in form of continuous positive airway pressure (CPAP), in which positive pressure is maintained in the airway throughout the respiratory cycle. Furthermore, the patient wears a mask over mouth and/or nose for continuous positive airway pressure (CPAP). However, current masks apply excessive pressure to the patient's face resulting in discomfort and possibly skin irritation. In addition, the current marks are inefficient to allow outflow of excessive carbon dioxide exhaled by the patient. Further, the current masks have ports and other devices on front face of respiratory face mask. Furthermore, the other devices include tubes, PEEP valves, filters, and the like. Moreover, the current masks with front ports cannot facilitate treatment of the patient lying down in prone or face down position due to obstruction caused by the front ports of the respiratory face mask and the other devices.

In light of the above-stated discussion, there is a need to overcome the above stated disadvantages.

### BRIEF SUMMARY OF THE INVENTION

In an aspect, the present disclosure provides the CPAP mask. The CPAP mask with side ports for providing continuous positively pressurized air to a patient. The CPAP mask includes a mask body, a plurality of strap holders, and a cushion seal. The mask body includes a left side wall, a left port, a front face, a right side wall, and a right port. In addition, the left side wall has a first through-hole. Further, the left port is on the left side wall. Furthermore, the front face of the CPAP mask is a transparent member. Moreover, the front face allows a plurality of medical practitioners to view one or more facial features of the patient. Also, the right side wall has a second through-hole. Also, the right port is on the right side wall. Also, the plurality of strap holders is on the left side wall and the right side wall of the mask body. Also, the plurality of strap holders clamps a medical mask strap. Also, the medical mask strap holds head of the patient with the CPAP mask. Also, the cushion seal is fastened along a perimeter of the CPAP mask. Also, the cushion seal connects the CPAP mask to facial area of the patient.

A primary object of the present disclosure is to provide a CPAP mask to provide continuous positively pressurized air to a patient.

Another objective of the present disclosure is to provide the CPAP mask to minimize pressure on face of the patient to provide comfort fitting.

Yet another object of the present disclosure is to provide the CPAP mask to allow outflow of excessive carbon dioxide exhaled by the patient.

Yet another object of the present disclosure is to provide the CPAP mask to enable a doctor to view features of the patient through transparent front face.

Yet another object of the present disclosure is to provide the CPAP mask with side ports to facilitate treatment of the patient lying in prone position or face down position

In an embodiment of the present disclosure, the CPAP mask securely covers nose and mouth of the patient.

In an embodiment of the present disclosure, the right port is connected with a ventilator through a flexible hose. In addition, the flexible hose allows continuous positive airway pressure ventilation for the patient. Further, the patient receives oxygenated air from the ventilator through the flexible hose.

In an embodiment of the present disclosure, the left port is aligned with the right port. In addition, the alignment enables unobstructed flow of the oxygenated air from the ventilator.

In an embodiment of the present disclosure, the cushion seal flexes according to shape of the facial area of the patient. In addition, the cushion seal seals the CPAP mask with the facial area of the patient.

In an embodiment of the present disclosure, the left port of the CPAP mask is connected to a PEEP valve and a HEPA filter unit. In addition, the PEEP valve maintains pressure of expiration air of the patient. Further, the HEPA filter unit traps a plurality of bacteria and viruses and a plurality of particulates. Furthermore, the HEPA filter unit filters outflow of the expiration air of the patient.

In an embodiment of the present disclosure, the CPAP mask is utilized for a plurality of positions. In addition, the patient rests in the plurality of positions. Further, the plurality of positions includes supine position and prone position.

### DESCRIPTION OF THE DRAWINGS

In order to best describe the manner in which the above-described embodiments are implemented, as well as define other advantages and features of the disclosure, a more particular description is provided below and is illustrated in the appended drawings. Understanding that these drawings depict only exemplary embodiments of the invention and are not therefore to be considered to be limiting in scope, the examples will be described and explained with additional specificity and detail through the use of the accompanying drawings in which:

FIG. 1 illustrates a first side view of a CPAP mask, in accordance with various embodiments of the present disclosure;

FIG. 2 illustrates a front view of the CPAP mask, in accordance with various embodiments of the present disclosure;

FIG. 3 illustrates a second side view of the CPAP mask, in accordance with various embodiments of the present disclosure;

FIG. 4 illustrates a third side view of the CPAP mask, in accordance with various embodiments of the present disclosure;

FIG. 5 illustrates a fourth side view of the CPAP mask with a cushion seal, in accordance with various embodiments of the present disclosure;

FIG. 6 illustrates a fifth side view of the CPAP mask with the cushion seal, in accordance with various embodiments of the present disclosure;

FIG. 7 illustrates a front view of a patient with the CPAP mask, a PEEP valve and a HEPA filter unit, in accordance with an embodiment of the present disclosure;

FIG. 8 illustrates a front view of the CPAP mask with the PEEP valve and the HEPA filter unit, in accordance with another embodiment of the present disclosure;

FIG. 9 illustrates a first side view of the HEPA filter unit, in accordance with an embodiment of the present disclosure;

FIG. 10 illustrates a second side view of the HEPA filter unit, in accordance with another embodiment of the present disclosure;

FIG. 11 illustrates a first side view of the PEEP valve, in accordance with an embodiment of the present disclosure; and

FIG. 12 illustrates a second side view of the PEEP valve, in accordance with another embodiment of the present disclosure.

It should be noted that the accompanying figures are intended to present illustrations of few exemplary embodiments of the present disclosure. These figures are not intended to limit the scope of the present disclosure. It should also be noted that accompanying figures are not necessarily drawn to scale.

### REFERENCE NUMERALS IN THE DRAWINGS

For a more complete understanding of the present invention parts, reference is now made to the following descriptions:
100. First side view.
102. CPAP mask.
104. Left side wall.
106. Left port.
108. Right port.
110. Front face.
112. Right side wall.
114. Plurality of strap holders.
116. Cushion seal.
118. PEEP valve.
120. HEPA filter unit.
122. Flexible hose.
124. Patient.
200. Front view.
300. Second side view.
400. Third side view.
500. Fourth side view.
600. Fifth side view.
700. Front view.
800. Front view.
900. First side view.
1000. Second side view.
1100. First side view.
1200. Second side view.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention.

Reference in this specification to “one embodiment” or “an embodiment” means that a particular feature, structure, or characteristic described in connection with the embodiment is included in at least one embodiment of the present technology. The appearance of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

Reference will now be made in detail to selected embodiments of the present disclosure in conjunction with accompanying figures. The embodiments described herein are not intended to limit the scope of the disclosure, and the present disclosure should not be construed as limited to the embodiments described. This disclosure may be embodied in different forms without departing from the scope and spirit of the disclosure. It should be understood that the accompanying figures are intended and provided to illustrate embodiments of the disclosure described below and are not necessarily drawn to scale. In the drawings, like numbers refer to like elements throughout, and thicknesses and dimensions of some components may be exaggerated for providing better clarity and ease of understanding.

Moreover, although the following description contains many specifics for the purposes of illustration, anyone skilled in the art will appreciate that many variations and/or alterations to said details are within the scope of the present technology. Similarly, although many of the features of the present technology are described in terms of each other, or in conjunction with each other, one skilled in the art will appreciate that many of these features can be provided independently of other features. Accordingly, this description of the present technology is set forth without any loss of generality to, and without imposing limitations upon, the present technology.

It should be noted that the terms “first”, “second”, and the like, herein do not denote any order, ranking, quantity, or importance, but rather are used to distinguish one element from another. Further, the terms “a” and “an” herein do not denote a limitation of quantity, but rather denote the presence of at least one of the referenced item.

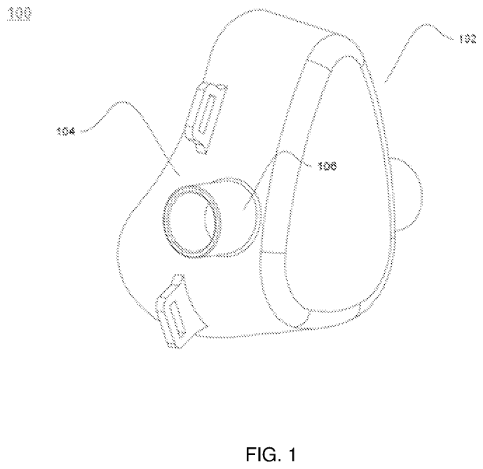

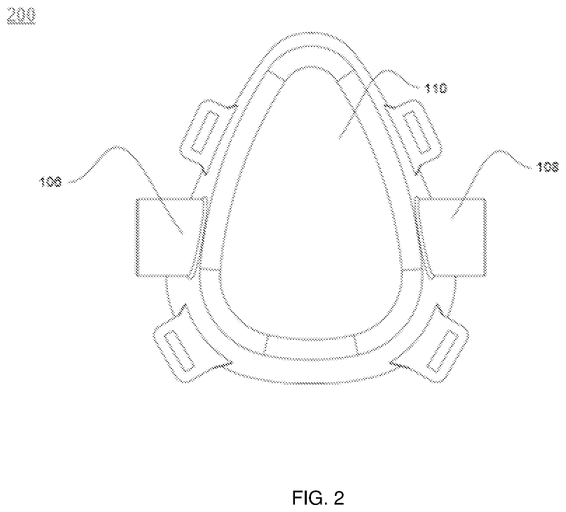

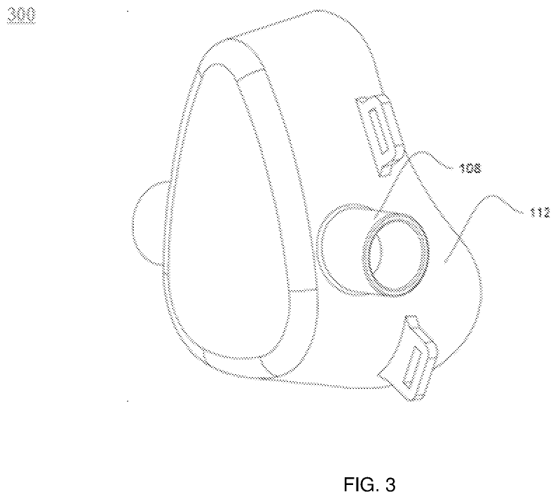

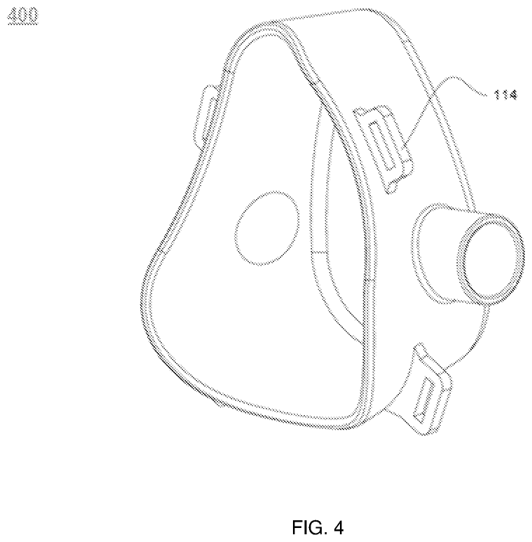

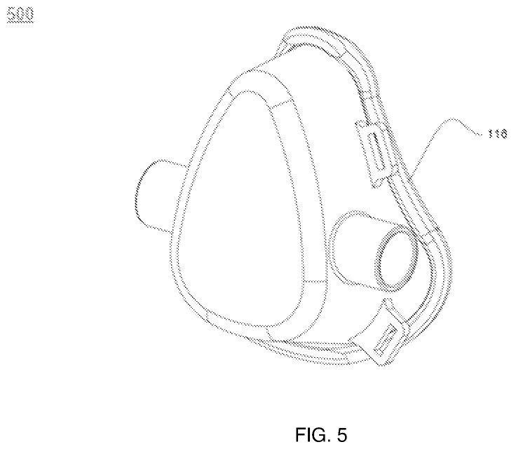

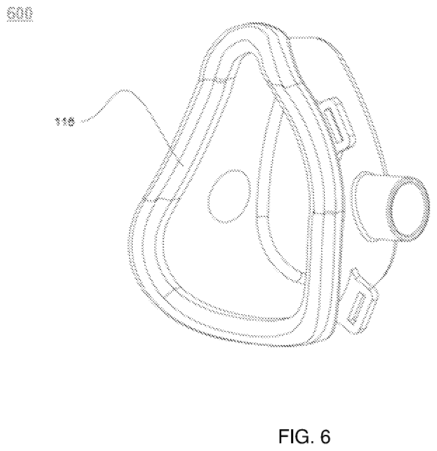

FIG. 1 illustrates a first side view 100 of the CPAP mask 102, in accordance with various embodiments of the present disclosure. FIG. 2 illustrates a front view 200 of the CPAP mask 102, in accordance with various embodiments of the present disclosure. FIG. 3 illustrates a second side view 300 of the CPAP mask 102, in accordance with various embodiments of the present disclosure. FIG. 4 illustrates a third side view 400 of the CPAP mask 102, in accordance with various embodiments of the present disclosure. FIG. 5 illustrates a fourth side view 500 of the CPAP mask 102 with a cushion seal 116, in accordance with various embodiments of the present disclosure. FIG. 6 illustrates a fifth side view 600 of the CPAP mask 102 with the cushion seal 116, in accordance with various embodiments of the present disclosure. In an embodiment of the present disclosure, the CPAP mask 102 is utilized for a plurality of positions. In addition, the patient 124 rests in the plurality of positions. Further, the plurality of positions includes supine position and prone position. In an embodiment of the present disclosure, the patient 124 lies in supine position. In another embodiment of the present disclosure, the patient 124 lies in prone position. The CPAP mask 102 has a cup-shaped mask body that fits over a person's nose and mouth.

The CPAP mask 102 with side ports provides continuous positively pressurized air to a patient 124. In addition, the CPAP mask 102 corresponds to a continuous positive airway pressure mask. Further, the CPAP mask 102 maintains the continuous positive airway pressure for the patient 124. In an embodiment of the present disclosure, the patient 124 is suffering from one or more respiratory disorders. In addition, the one or more respiratory disorders include but may not be limited to asthma, chronic obstructive pulmonary disease (COPD), pulmonary fibrosis, sleep apnea, pneumonia, lung cancer, and Coronavirus (COVID-19). In an embodiment of the present disclosure, the patient 124 suffers from a respiratory disorder of the one or more respiratory disorders. In addition, the respiratory disorder of the one or more respiratory disorders causes inflammation on internal walls of lung due to a plurality of viruses. Further, the plurality of viruses include but may not be limited to COVID19 virus, SARS virus, MERS, and Influenza virus. Furthermore, the patient 124 requires a non-invasive form of respiratory support. In an embodiment of the present disclosure, the non-invasive form of respiratory support is CPAP. In addition, CPAP corresponds to continuous positive airway pressure. Further, the CPAP mask 102 securely covers nose and mouth of the patient 124. In an example, a patient P1 is suffering from acute pneumonia due to Corona Virus. In addition, the patient P1 is not able to breathe. Further, a CPAP mask M1 connected with a ventilator V1 is utilized to assist the patient P1.

The CPAP mask 102 includes a mask body, a plurality of strap holders 114, and the cushion seal 116. The mask body includes a left side wall 104, a left port 106, a front face 110, a right side wall 112, and a right port 108. The mask body is a cup-shaped body of the CPAP mask 102 that fits on face of the patient 124. The mask body has hollow space to cover mouth and nose of the patient 124. In addition, the left side wall 104 has a first through-hole. Further, the first through-hole is provided to facilitate the left port 106. In an embodiment of the present disclosure, number of the plurality of strap holders 114 on the left side wall 104 is 2. In another embodiment of the present disclosure, number of the plurality of strap holders 114 on the left side wall 104 may vary. In addition, the left port 106 is on the left side wall 104.

In an embodiment of the present disclosure, the front face 110 of the CPAP mask 102 is a transparent member. In addition, the front face 110 allows a plurality of medical practitioners to view one or more facial features of the patient 124. Further, the one or more medical practitioners include but may not be limited to doctors, nurses, clinical assistant, patient services assistant, and porter. Furthermore, the front face 110 creates an internal breathing chamber for the patient 124. In an example, a patient P1 might be suffering from Covid-19. In addition, a doctor D1 puts a CPAP mask M1 covering mouth and nose of the patient P1. Further, the doctor D1 views breathing difficulty, opened mouth, and narrower maxillae of the patient P1 through transparent member of the CPAP mark M1.

The right side wall 112 has a second through-hole. In addition, the second through-hole is provided to facilitate the right port 108. In an embodiment of the present disclosure, number of the plurality of strap holders 114 on the right side wall 112 is 2. In another embodiment of the present disclosure, number of the plurality of strap holders 114 on the right side wall 112 may vary. In addition, the right port 108 is on the right side wall 112. Further, the left port 106 is aligned with the right port 108. Furthermore, the alignment enables unobstructed flow of oxygenated air from a ventilator. Moreover, the left port 106 and the right port 108 are the side ports of the CPAP mask 102. In general, ventilator supplies breathable air. In addition, the ventilator delivers the breathable air to patient who is physically unable to breathe.

The plurality of strap holders 114 is on the left side wall 104 and the right side wall 112. In addition, the plurality of strap holders 114 clamps a medical mask strap. In an embodiment of the present disclosure, the medical mask strap holds head of the patient 124 with the CPAP mask 102. In another embodiment of the present disclosure, the medical mask strap wraps around ear of the patient 124. In an embodiment of the present disclosure, the medical mask strap is made up of cloth fabric. In another embodiment of the present disclosure, the medical mask strap may be made up of any other type of fabric. In yet another embodiment of the present disclosure, the medical mask strap is made up of rubber. In yet another embodiment of the present disclosure, the medical mask strap is made up of plastic. In yet another embodiment of the present disclosure, the medical mask strap may be made up of any other material.

The cushion seal 116 is fastened along a perimeter of the CPAP mask 102. The perimeter is a path that surrounds a two-dimensional shape of the CPAP mask 102 that touches facial area of the patient 124. The perimeter of the CPAP mask 102 is covered with the cushion seal 116 to provide protection and comfort to the patient 124. In addition, the cushion seal 116 connects the CPAP mask 102 to facial area of the patient 124. In an embodiment of the present disclosure, the cushion seal 116 provides cushioning to facial area of the patient 124 in prone position. In another embodiment of the present disclosure, the cushion seal 116 provides cushioning to facial area of the patient 124 in supine position. In addition, the cushion seal 116 flexes according to shape of the facial area of the patient 124. Further, the cushion seal 116 seals the CPAP mask 102 with the facial area of the patient 124. In an embodiment of the present disclosure, the cushion seal 116 is made up of a thick medical grade soft rubber. In another embodiment of the present disclosure, the cushion seal 116 is made up of a silicone elastomer. In addition, the cushion seal 116 forms air chamber between the facial area of the patient 124 and the CPAP mask 102.

The CPAP mask 102 allows the patient 124 to perform a plurality of activities. In addition, the patient 124 is comfortable wearing the CPAP mask 102. Further, the plurality of activities includes but may not be limited to reading books, watching Television, playing play station and sleeping in prone position.

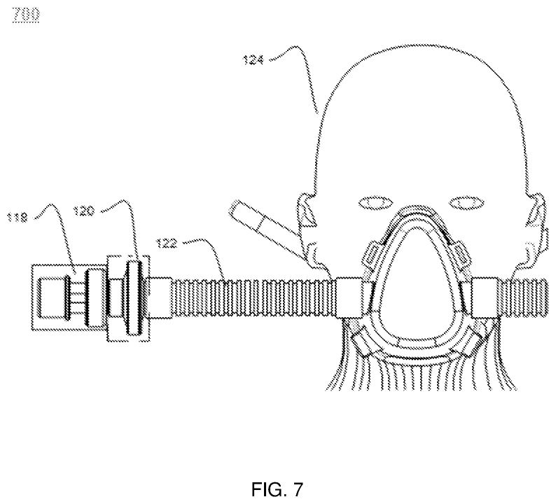

FIG. 7 illustrates a front view 700 of a face of the patient 124 with the CPAP mask 102, a PEEP valve 118 and a HEPA filter unit 120, in accordance with an embodiment of the present disclosure. The front view 700 includes the patient 124, the CPAP mask 102, the PEEP valve 118, the HEPA filter unit 120, and a flexible hose 122. In addition, the right port 108 is connected with the ventilator through the flexible hose 122. Further, the flexible hose 122 allows continuous positive airway pressure ventilation for the patient 124. Furthermore, the patient 124 receives the oxygenated air from the ventilator through the flexible hose 122. In an embodiment of the present disclosure, the left port 106 is connected with the flexible hose 122. In addition, the PEEP valve 118 and the HEPA filter unit 120 are connected to the left port 106 of the CPAP mask 102 through the flexible hose 122. Further, the PEEP valve 118 maintains pressure of expiration air of the patient 124. Furthermore, the HEPA filter unit 120 contains a plurality of bacteria and viruses and a plurality of particulates. Moreover, the HEPA filter unit 120 filters outflow of the expiration air of the patient 124.

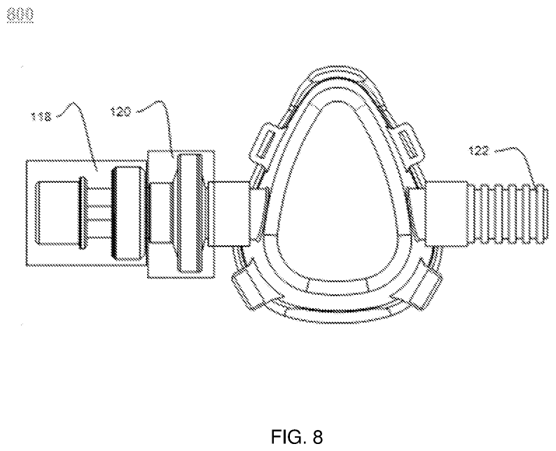

FIG. 8 illustrates a front view 800 of the CPAP mask 102 with the PEEP valve 118 and the HEPA filter unit 120, in accordance with another embodiment of the present disclosure. The front view 800 includes the CPAP mask 102, the PEEP valve 118, the HEPA filter unit 120, and the flexible hose 122. In addition, the right port 108 is connected with the ventilator through the flexible hose 122. Further, the flexible hose 122 allows continuous positive airway pressure ventilation for the patient 124. Furthermore, the patient 124 receives the oxygenated air from the ventilator through the flexible hose 122. Moreover, the PEEP valve 118 and the HEPA filter unit 120 are connected to the left port 106 of the CPAP mask 102 directly. Also, the PEEP valve 118 maintains pressure of expiration air of the patient 124. Also, the HEPA filter unit 120 contains the plurality of bacteria and viruses and the plurality of particulates. Also, the HEPA filter unit 120 filters outflow of the expiration air of the patient 124.

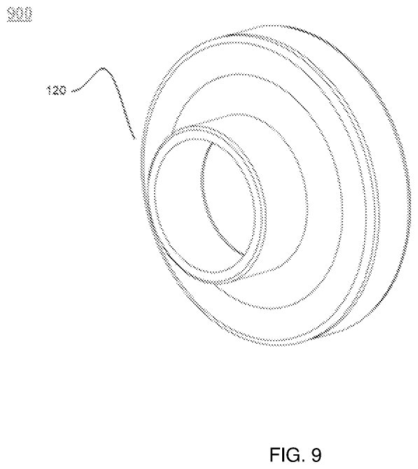

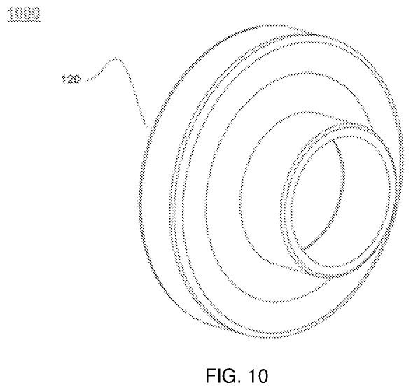

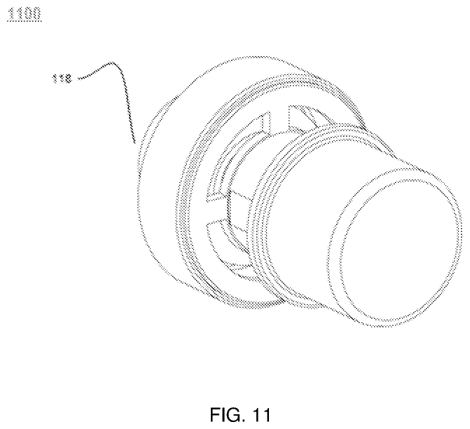

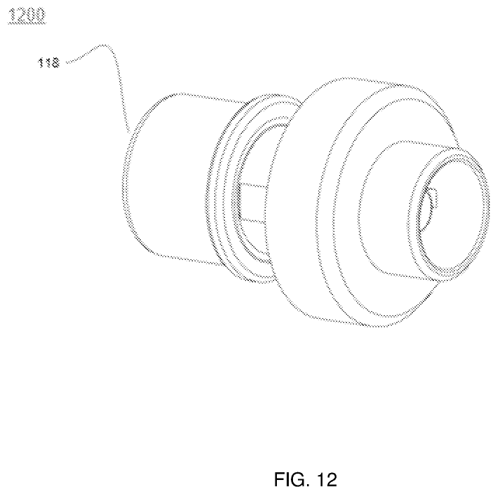

FIG. 9 illustrates a first side view 900 of the HEPA filter unit 120, in accordance with an embodiment of the present disclosure. FIG. 10 illustrates a second side view 1000 of the HEPA filter unit 120, in accordance with another embodiment of the present disclosure. FIG. 11 illustrates a first side view 1100 of the PEEP valve 118, in accordance with an embodiment of the present disclosure. FIG. 12 illustrates a second side view 1200 of the PEEP valve 118, in accordance with another embodiment of the present disclosure. The HEPA filter unit 120 corresponds to high-efficiency particulate air filter unit. In addition, the HEPA filter unit 120 includes a high energy ultra-violet light unit. Further, the high energy ultra-violet light unit eliminates the plurality of bacteria and viruses. Furthermore, the HEPA filter unit 120 provides protection against airborne disease transmission. Moreover, the HEPA filter unit 120 traps pollen and dust mites to protect the patient 124.

The PEEP valve 118 corresponds positive end expiratory pressure valve. In addition, the PEEP valve 118 is utilized to maintain pressure on lower airways at end of breathing cycle. In an embodiment of the present disclosure, PEEP valve 118 has fixed value colour coded valves made from a transparent material that allows monitoring of respiratory rate of the patient 124. In addition, the HEPA filter unit 120 has an inner cylindrical portion and an external cylindrical portion. Further, the PEEP valve 118 has an inner cylindrical portion and an external cylindrical portion. Furthermore, the inner cylindrical portion of the HEPA filter unit 120 connects with cylindrical portion of the left port 106. Moreover, the external cylindrical portion of the HEPA filter unit 120 connects with the inner cylindrical portion of the PEEP valve 118.

The present invention has various advantages over the prior art. The present invention provides a CPAP mask to provide continuous positively pressurized air to a patient. In addition, the present disclosure provides the CPAP mask to minimize pressure on face of the patient resulting in comfort. Further, the present disclosure provides the CPAP mask to allow outflow of excessive carbon dioxide exhaled by the patient. Furthermore, the present disclosure provides the CPAP mask to enable a doctor to view features of the patient through transparent front face. Moreover, the present disclosure provides the CPAP mask with side ports to facilitate treatment of the patient lying in prone position or face down position.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

Although the present disclosure has been explained in relation to its preferred embodiment(s) as mentioned above, it is to be understood that many other possible modifications and variations can be made without departing from the spirit and scope of the inventive aspects of the present invention. It is, therefore, contemplated that the appended claim or claims will cover such modifications and variations that fall within the true scope of the invention.

What is claimed is:

1. A CPAP mask with side ports for providing continuous positively pressurized air to a patient, the CPAP mask comprising:
a mask body, wherein the mask body comprising:
a left side wall, wherein the left side wall has a first through-hole;
a left port, wherein the left port is on the left side wall;
a front face, wherein the front face of the CPAP mask is a transparent member, wherein the front face allows a plurality of medical practitioners to view one or more facial features of the patient;
a right side wall, wherein the right side wall has a second through-hole;
a right port, wherein the right port is on the right side wall;
a plurality of strap holders, wherein the plurality of strap holders is provided on the left side wall and the right side wall of the mask body, wherein the plurality of strap holders clamps a medical mask strap, wherein the medical mask strap holds head of the patient with the CPAP mask; and,
a cushion seal, wherein the cushion seal is fastened along a perimeter of the CPAP mask, wherein the cushion seal connects the CPAP mask to facial area of the patient.

2. The CPAP mask as claimed in claim 1, wherein the CPAP mask securely covers nose and mouth of the patient.

3. The CPAP mask as claimed in claim 1, wherein the right port is connected with a ventilator through a flexible hose, wherein the flexible hose allows continuous positive airway pressure ventilation for the patient, wherein the patient receives oxygenated air from the ventilator through the flexible hose.

4. The CPAP mask as claimed in claim 1, wherein the left port is aligned with the right port, wherein the alignment enables unobstructed flow of the oxygenated air from the ventilator.

5. The CPAP mask as claimed in claim 1, wherein the cushion seal flexes according to shape of the facial area of the patient, wherein the cushion seal seals the CPAP mask with the facial area of the patient.

6. The CPAP mask as claimed in claim 1, wherein the left port of the CPAP mask is connected to a PEEP valve and a HEPA filter unit, wherein the PEEP valve maintains pressure of expiration air of the patient, wherein the HEPA filter unit traps a plurality of bacteria and viruses and a plurality of particulates, wherein the HEPA filter unit (120) filters outflow of the expiration air of the patient.

7. The CPAP mask as claimed in claim 1, wherein the CPAP mask is utilized for a plurality of positions, wherein the patient rests in the plurality of positions, wherein the plurality of positions comprising supine position and prone position.

8. A CPAP mask comprising:
a mask body capable of fitting over a facial area of a patient, wherein the mask body comprising:
a left side wall, wherein the left side wall has a left port;
a right side wall, wherein the right side wall has a right port;
a top wall;
a bottom wall; and
a front face connected to the left side wall, the right side wall, the top wall and the bottom wall, wherein the front face has no ports.

9. The CPAP mask as claimed in claim 8, wherein the front face of the CPAP mask is a transparent member allowing a view of facial features of the patient.

10. The CPAP mask as claimed in claim 8 further comprising a plurality of strap holders, wherein the plurality of strap holders is provided on the left side wall and the right side wall of the mask body, wherein the plurality of strap holders clamps a medical mask strap.

11. The CPAP mask as claimed in claim 8 further comprising a cushion seal fastened along a perimeter of the CPAP mask, wherein the cushion seal engages the CPAP mask to a facial area of the patient.

12. The CPAP mask as claimed in claim 8, wherein the CPAP mask securely covers nose and mouth of the patient.

13. The CPAP mask as claimed in claim 8, wherein the right port is connected to a ventilator through a flexible hose, wherein the flexible hose allows continuous positive airway pressure ventilation for the patient, wherein the patient receives oxygenated air from the ventilator through the flexible hose.

14. The CPAP mask as claimed in claim 8, wherein the left port and the right port are aligned to enable unobstructed flow of the oxygenated air from the ventilator.

15. The CPAP mask as claimed in claim 8, wherein the left port of the CPAP mask is connected to a PEEP valve and a HEPA filter unit, wherein the PEEP valve maintains pressure of expiration air of the patient, wherein the HEPA filter unit traps a plurality of bacteria and viruses and a plurality of particulates, wherein the HEPA filter unit filters outflow of the expiration air of the patient.

16. The CPAP mask as claimed in claim 8, wherein the CPAP mask is utilized in one or more positions such as a supine position and a prone position.

17. A CPAP mask comprising:
a mask body capable of fitting over a facial area of a patient, wherein the mask body comprising:
a left side wall, wherein the left side wall has a left port;
a right side wall, wherein the right side wall has a right port, wherein the left port and the right port are aligned to enable unobstructed flow of air;
a top wall;
a bottom wall; and
a front face connected to the left side wall, the right side wall, the top wall and the bottom wall.

18. The CPAP mask as claimed in claim 17, wherein the front face of the CPAP mask is a transparent member allowing a view of facial features of the patient.

19. The CPAP mask as claimed in claim 17, wherein the front face has no ports.
